(19) United States
(12) Patent Application Publication
(10) Pub. No.: US 2009/0090367 A1
(43) Pub. Date: Apr. 9, 2009

(54) Method of contraception

(76) Inventor: Scott B. Hurley, Chester, NJ (US)

Correspondence Address: WOLFF & SAMSON, P.C., ONE BOLAND DRIVE, WEST ORANGE, NJ, 07052, US

(21) Appl. No.: 11/906,588

(22) Filed: Oct. 3, 2007

(51) Int. Cl. A61F 6/06

(52) U.S. Cl. 128/830

(57) ABSTRACT

A contraceptive method including the steps of using a primary contraceptive and administering a secondary, immunocontraceptive to boost overall efficacy of the two contraceptives.

### BACKGROUND OF THE INVENTION

The goal of safe, reliable, reversible contraception has been the driver of most contraceptive options and methods development for centuries. Women and men want a method that is highly effective, reversible, and safe. Unfortunately, today's most popular methods are not 100% effective and carry risks. These methods include rhythm, diaphragms, spermicides, oral contraceptives, IUDs, condoms and hormonal implants.

Rhythm, diaphragms, condoms and spermicides all have low efficacy rates approaching 80-90% while oral contraceptives, IUDs and hormonal implants with higher efficacy rates have related safety issues associated with them.

With regard to oral contraceptive clinical trials, contraceptive efficacy approaches 100% with pearl indices (pregnancies per 100 women years of use) of between 3 and less than 1. Efficacy is high because oral contraceptives utilize three separate mechanisms of action to provide efficacy. They suppress ovulation, they thicken the cervical mucus, and they make the endometrial lining hostile to implantation. However, clinical trial efficacy does not include women who miss more than 2 pills or women who fail to start their new pill pack on the scheduled start day or fail to take their pills as directed. Also, any pregnancies which are attributable to other causes such as illnesses with vomiting or concomitant medication use outside of the study protocol are removed from the efficacy calculations. Studies looking at real world use, as opposed to controlled clinical trials, of oral contraceptives actually document a much lower efficacy rate of around 90 to 96% as opposed to the controlled clinical trial rates of 97% to nearly 100% due to user method failure, among other causes. This results in unintended pregnancies and many abortions.

To improve the safety profile of oral contraceptives the dosage of the hormones has been reduced in the formulations. This has resulted in a slight rise in pearl indices and a lower margin of error for pill taking on schedule.

Immunocontraceptive development, commonly referred to as “vaccines” has been known for decades. The goal of this development was to produce a long-term, safe, reversible method of contraception that would reduce the likelihood of user method failure, drug/drug and food/stomach interaction events from the unintended pregnancy equation. However, in clinical trials it was shown that while in most women the antibody titer levels were sufficient to achieve an expected efficacy rate of 100%, there were some women for whom the titer levels would provide an expected efficacy rate of only about 80%. Since no one could tell why some women's titers were lower than others and did not stay high for a long enough period of time (one year) this method of contraception was found to be unreliable and unacceptable and never made it onto the market as a method of human contraception. Reliability, predictability and reproducibility were all efficacy issues hindering the development of immunocontraceptive agents as a method of contraception.

Many immunocontraceptives are known in the art for both human and animal use. For example, U.S. Pat. No. 4,123,519 discloses an injectable contraceptive vaccine. Another contraceptive vaccine is disclosed in U.S. Pat. No. 5,637,300.

Contraceptive usage remains high but there is a need to find a way to improve the efficacy of these methods in real world use. There is a need to reduce the risk of unintended pregnancies caused by user method failure, drug/drug interactions, and poor drug blood level obtainment issues caused by drug/drug interactions and poor absorption from the GI tract. Also there is a need to continue reducing a woman's exposure to contraceptive hormones for safety reasons while keeping the efficacy of the contraceptive method near 100%.

### SUMMARY AND DESCRIPTION OF THE INVENTION

The object of the present invention is to address the above-discussed problems with the prior art.

This object is achieved by a contraception method having a concomitant use of an immunocontraceptive agent with another method of contraception to boost the real world efficacy rate of the primary contraceptive method to between 95% and 100% efficacy. The efficacy rate of this concomitant use will be better than either the primary method of use or the use of the immunocontraceptive agent alone. The immunocontraceptive agent acts as a backup or additional method of contraception should the primary method fail and will/may improve the safety profile of the primary contraceptive method by allowing for reduced exposure to the primary method's ingredients.

Since the immunocontraceptive is being added to a primary method of contraception to boost its real world efficacy, the patient variability seen in the antigen titer levels with immunocontraceptive use are acceptable and will reduce the patient's efficacy failure rate 80 to 100%. A highly desirable outcome.

The immunocontraceptive agent is administered by injection, nasally, orally or topically on a regular time frame as determined by clinical trials. This time frame will be from between once a month to once every five years.

The immunocontraceptive agent is labeled as an adjunct, secondary or backup method of contraception to the users' primary method of contraception.

The immunocontraceptive agent may be administered prior to, or during the patient's use of their primary contraceptive method.

It will be packaged as a single use dose, multiple dose vial, or starter package with or without a primary method of contraception.

The concomitant use of the immunocontraceptive agent with the primary method of contraception will reduce the real world failure rate of the primary method by about at least 80%. For example: An oral contraceptive with a real world failure rate of 8% will now have a real world failure rate of just 1.6% or in other terms; the real world efficacy of the oral contraceptive will go from 92% to 98.4%. This will result in a substantial reduction in the number of unintended pregnancies and resulting abortions.

The primary method of contraception which is boosted by use of an immunocontraceptive agent can be any one of the known conventional contraceptive methods, including but not limited to, diaphragms, spermicides, oral contraceptives, IUDs, condoms and hormonal implants.

The specific type of immunocontraceptive agent used is not of importance relative to the novelty of the present invention. What is of significance is the combination of an immunocontraceptive with a primary contraceptive to provide an overall improved efficacy.

Although the present invention has been described in relation to particular embodiments thereof, many other variations and modifications and other uses will become apparent to those skilled in the art. It is preferred, therefore, that the present invention be limited but by the specific disclosure herein, but only by the appended claims.

1. A contraceptive method, comprising the steps of:
using a primary contraceptive; and
administering a secondary, immunocontraceptive to boost overall efficacy of the contraceptives.

2. The contraceptive method according to claim 1, including administering an oral contraceptive as the primary contraceptive.

3. The contraceptive method according to claim 1, including using a diaphragm as the primary contraceptive.

4. The contraceptive method according to claim 1, including using an IUD as the primary contraceptive.

5. The contraceptive method according to claim 1, including using hormonal implants as the primary contraceptive.

6. The contraceptive method according to claim 1, including administering the immunocontraceptive by injection.

7. The contraceptive method according to claim 1, including administering the immunocontraceptive orally.

8. The contraceptive method according to claim 1, including administering the immunocontraceptive topically.

9. The contraceptive method according to claim 1, including administering the immunocontraceptive on a regular time frame.

10. The contraceptive method according to claim 9. wherein the time frame is between once a month and once every five years.

11. The contraceptive method according to claim 1, further including labeling the immunocontraceptive as a secondary contraceptive to the primary contraceptive.

12. The contraceptive method according to claim 1, including administering the immunocontraceptive prior to use of the primary contraceptive.

13. The contraceptive method according to claim 1, including administering the immunocontraceptive during use of the primary contraceptive.

14. The contraceptive method according to claim 1, further including packaging the primary and secondary contraceptives together.

15. The contraceptive method according to claim 1, further including packaging the secondary contraceptive as a single use dose.

16. The contraceptive method according to claim 1, further including packaging the secondary contraceptive as a multiple dose vial.

17. The contraceptive method according to claim 1, further including packaging the secondary contraceptive as a starter package with or without the primary contraceptive.
